SEQUENCE LISTING (7 sequences)

SEQ ID NO: 1 - DNA, 119 bp, Artificial Sequence
  consensus sequence
atcagtagaattggatgctcctaattaccactatgaggagtgctcctgttatcctgatgccggcgaaattacatgtgtgtgcagggataattggcatggttcaaataggccatgggtat

SEQ ID NO: 2 - DNA, 119 bp, Artificial Sequence
  consensus sequence
atcagtcgaattggatgctcccaattatcactatgaggaatgctcctgttatcctgatgccggcgaaatcacatgtgtgtgcagggataattggcatggctcaaatcggccatgggtat

SEQ ID NO: 3 - DNA, 122 bp, Artificial Sequence
  consensus sequence
atcagtcgaattggatgctcctaattatcactatgaggagtgctcctgttatccttttgatgccggcgaaatcacatgtgtgtgcagggataattggcatggctcaaataggccatgggtat

SEQ ID NO: 4 - DNA, 122 bp, Artificial Sequence
  consensus sequence
atcagtcgaattggatgctcctaattatcactatgaggagtgctcctgttatcctnnngatgccggcgaaatcacatgtgtgtgcagggataattggcatggctcaaataggccatgggtat

SEQ ID NO: 5 - DNA, 20 bp, Artificial Sequence
  primer sequence
agtcgaattggatgctccta

SEQ ID NO: 6 - DNA, 18 bp, Artificial Sequence
  primer sequence
cccatggcctatttgagc

SEQ ID NO: 7 - DNA, 29 bp, Artificial Sequence
  probe sequence
ggataacaggagcactcctcatagtgata

(19) United States
(12) Patent Application Publication
(10) Pub. No.: US 2013/0273521 A1
(43) Pub. Date: Oct. 17, 2013

(54) SIGNALLING SYSTEM

(71) Applicants: Martin Lee, Salisbury Wiltshire (GB); Mark Laverick, Salisbury Wiltshire (GB)

(72) Inventors: Martin Lee, Salisbury Wiltshire (GB); Mark Laverick, Salisbury Wiltshire (GB)

(73) Assignee: ENIGMA DIAGNOSTICS LIMITED, Salisbury Wiltshire (GB)

(21) Appl. No.: 13/697,291

(22) PCT Filed: May 11, 2011
(86) PCT No.: PCT/GB2011/050903

§ 371 Date: Jul. 2, 2013

(30) Foreign Application Priority Data
May 11, 2010 (GB) 1007867.3

(51) Int. Cl. C12Q 1/68; C12Q 1/70

(52) U.S. Cl. CPC C12Q 1/6818; C12Q 1/701
USPC 435/5; 435/6.11; 536/24.33

(57) ABSTRACT

The invention concerns a system for detecting a target nucleic acid molecule of a particular sequence in a sample, said system comprising (i) an oligonucleotide primer complementary to a said target nucleic acid molecule, which primer has no internal complementarity, is able to amplify said target sequence and carries a first label linked to said oligonucleotide at its 5′ end; and (ii) an oligonucleotide probe which carries a second label that is able to interact with said first label to produce a detectable signal, wherein the oligonucleotide probe binds an extension product of said primer such that the first and second label can interact to produce a detectable signal. Methods for using said system in particular in a nucleic acid assay, kits comprising the system and elements of it form a further aspect of the invention.

### FIELD OF INVENTION

The present invention provides a system for detecting or quantifying nucleic acid molecules, use of said system in assays such as a dual hybridisation assay; as well as kits and methods that utilise the system.

### BACKGROUND

Labelled oligonucleotides, such as probes or primers, for detecting a target sequence within a DNA molecule are known. They typically include a light emitting label such as a fluorescent label and may make use of fluorescence energy transfer (FET) or fluorescence resonance energy transfer (FRET).

In FET one or more nucleic acid probes are labelled with fluorescent molecules, one of which acts as an energy donor molecule and the other of which acts as an energy acceptor molecule. These are sometimes known as a reporter molecule and a quencher molecule, respectively. The donor molecule is excited with a specific wavelength of light which falls within its excitation spectrum and, subsequently, it emits light within its fluorescence emission spectrum. The compatible acceptor molecule is excited at this wavelength by accepting energy from the donor molecule by a variety of distance-dependent energy transfer mechanisms. A specific example of fluorescence energy transfer which can occur is Fluorescence Resonance Energy Transfer or “FRET”. Generally, the acceptor molecule accepts the emission energy of the donor molecule when they are in close proximity (e.g., on the same, or a neighbouring molecule). The basis of fluorescence energy transfer detection is to monitor the changes at donor and acceptor emission wavelengths.

Examples of molecules used as donor and/or acceptor molecules in FRET systems include, amongst others, SYBRGold, SYBRGreenI, Fluorescein, rhodamine, Cy5, Cy5.5 and ethidium bromide, as well as others such as SYTO dyes as listed, for example, in WO2007/093816.

There are two commonly used types of FET or FRET probes, those using hydrolysis of nucleic acid probes to separate donor from acceptor and those using hybridisation to alter the spatial relationship of donor and acceptor molecules.

Known fluorescence polymerase chain reaction (PCR) monitoring techniques include both these types of probes in PCR thermal cycling devices. The reactions are carried out homogeneously in a closed tube format on thermal cyclers. Reactions are monitored using a fluorimeter. The precise form of the assays varies but often relies on FET between two fluorescent moieties within the system in order to generate a signal indicative of the presence of the product of amplification.

Typically, fluorescence increases due to a rise in the bulk concentration of DNA during amplifications. This increase in fluorescence can be used to measure reaction progress and to determine the target molecule copy number. Furthermore, by monitoring fluorescence with a controlled change of temperature, DNA melting curves can be generated, for example, at the end of PCR thermal cycling. The melting temperature of a DNA duplex depends on its base composition and length. All PCR products for a particular primer pair should have the same melt temperature unless there is mispriming, primer-dimer artefacts or some other problem. Melt temperature data can be used, therefore, to determine the specificity of the probes/purity of the amplified DNA.

Hybridisation probes are available in a number of forms. Molecular beacons are oligonucleotides that have complementary 5′ and 3′ sequences such that they form hairpin loops. Terminal fluorescent labels are in close proximity for FRET to occur when the hairpin structure is formed. Following hybridisation of molecular beacons to a complementary sequence the fluorescent labels are separated so FRET does not occur, forming the basis of detection.

Pairs of labelled oligonucleotides may also be used (a dual hybridisation system). These hybridise in close proximity to one another on a PCR product strand bringing donor and acceptor molecules (e.g., fluorescein and rhodamine) together so that FRET can occur, as disclosed in WO97/46714, for example. Enhanced FRET is the basis of detection. The use of two probes requires the presence of a reasonably long known sequence so that two probes which are long enough to bind specifically can bind in close proximity to each other. This can be a problem in some diagnostic applications, where the length of conserved sequences in an organism which can be used to design an effective probe, such as the HIV virus, may be relatively short.

Furthermore, the use of pairs of probes involves more complex experimental design. For example, a signal provided by the melt of a probe is a function of the melting-off of both probes. Therefore, two separately labelled probes are required for the detection of each single sequence.

A variation of this type of system is shown in FIGS. 1 and 2 below and uses a labelled amplification primer with a single adjacent probe as the two elements of the signalling system. This is as disclosed in WO97/46714 and is generally referred to as a trans dual hybridisation system. However, such a system can be used to detect a target sequence which is relatively close to the site of the binding of the amplification primer, since the label on the probe and the label on the primer must be in sufficient proximity when the probe is bound for FRET to occur.

Although the probe may be labelled at its 3′ end, to ensure that it is brought in close proximity to the label on the primer, the primer may not carry a label at the 3′ end as this may block or impede extension thereof during the amplification. Therefore, the primer oligonucleotide in these assays is labelled internally with its label away from or remote from either of its ends. This ensures that the 3′ end remains free for extension whilst the distance over which FET or FRET is required to occur is minimised in order to generate a strong signal.

Dual hybridisation assays can form part of a multiplex arrangement where more than one segment of DNA is detected using more than one set of primers and associated probes. In this complex arrangement, labels are selected so as to allow discrimination and so, typically, each label emits a signal at a distinct and different wavelength to those of other labels used within the system. In multiplex PCR it is conventional to label the probes with a donor molecule, which may be common to all probes.

The convention in real-time assays using a ‘Universal Donor’ arrangement, where a single donor molecule interacts with a range of different acceptors, is to use the acceptor molecule as the reporter. Therefore, the probe is labelled with an acceptor molecule and this latter arrangement is shown in FIG. 1.

However, the applicants have found that the reporter molecule on the probe molecule can be subject to proximal quenching as a result of the interaction with the nascent strand itself (a “LUX” effect, so called because proximal quenching is exploited in the Lux® fluorogenic methods). Similarly, an internally located donor molecule on the primer may also be subject to some quenching as a result of interaction with adjacent nucleotides in the primer. This gives rise to a change in spectral emission, which is detrimental in particular in the context of a multiplex analysis where signal integrity is paramount if one is to distinguish between a range of, sometimes, closely related signals.

The seminal report covering a primer probe FRET arrangement (Bernard et al. (1998 Anal. Biochem. 255 101-107) states that a FRET dye pair such as FITC and Cy5 is used. The FITC donor is placed on the 3′ end of the probe that binds to the nascent strand generated by the primer. The primer is internally labelled with the Cy5 acceptor. This means that whilst an acceptor molecule on the primer may undergo proximal quenching, it is not sufficiently excited by the instrument illumination to contribute to the specific signal. This arrangement is shown in FIG. 2.

These workers reported the internal labelling of the primer with the acceptor molecule to be critical to the signalling efficiency of the FRET pair. This may be because of a need to reduce the distance over which FRET occurs. However, internal labelling of oligonucleotides is expensive and difficult and not all labels are available for application using the methods necessary to achieve this. Therefore, such assays are costly and the options for multiplexing are reduced.

The present inventors have, therefore, been investigating how to operate a trans dual hybridisation assay which can suitably be used in a multiplex arrangement whilst maintaining a ‘Universal Donor’ arrangement, being simple and of a low cost to manufacture.

### SUMMARY OF INVENTION

According to a first aspect of the invention, there is provided a system for detecting a target nucleic acid molecule of a particular sequence in a sample, said system comprising

(i) an oligonucleotide primer complementary to a said target nucleic acid molecule, which primer has no internal complementarity, is able to amplify said target sequence and carries a first label linked to said oligonucleotide at its 5′ end; and

(ii) an oligonucleotide probe which carries a second label that is able to interact with said first label to produce a detectable signal, wherein the oligonucleotide probe binds an extension product of said primer such that the first and second label can interact to produce a detectable signal.

The term “primer has no internal complementarity” indicates that regions of the primer will not bind or associate with other regions in the same primer, i.e., no region of the primer is complementary to any other region of the primer. Such self-complementary primers are required in some other systems, such as that disclosed in EP-A-0566751.

The applicants have found that there is no need to provide the label internally on the primer, but that a detectable interaction can occur even if the primer carries the first label at the 5′ end thereof.

In an embodiment of the invention, one of the first or second labels comprises a donor molecule of a FET or FRET signalling system and the other is an acceptor of the FET or FRET signalling system. Accordingly, one of said labels is a fluorescence donor molecule and the other is a fluorescence acceptor molecule able to absorb fluorescence from the donor molecule. Particular combinations of donor and acceptor molecules that are able to interact are known in the art and it is a matter of routine to check that any particular combination will operate in the system of the present invention. In particular, however, the applicants have found that fluorescein or a fluorescein derivative such as FAM may be used as a fluorescent energy donor in the system of the invention. Suitable acceptor or reporter labels will include any dyes able to accept energy from the selected donor such as FAM. These include TYE™ dyes such as TYE563, TEX615, TYE 665, TYE700, TYE705 available from Integrated DNA Technologies, (IDT) Iowa, as well as Cy3, Cy3.5, Cy5, Cy5.5, Texas Red, ROX and rhodamine.

In one embodiment, the first label is linked to the 5′ end of the primer by way of a spacer group. Such groups are believed to enhance any dynamic or Dexter interaction (Real-time PCR: Current Technology and Applications (ISBN: 10-1904455395) Eds Julie Logan, Kirstin Edwards, Nick Saunders; chapter 3: Lee et al.). This may compensate, at least to some extent, for any reduction in Forster interaction between the first and second labels as a result of the increased distance between them. In addition, it further reduces any proximal quenching effects exerted on the first label by adjacent nucleotides within the primer itself.

Suitable spacers including phosphoramidate spacers including for example from 3-10 carbon atoms such as a three carbon spacer, or “C3 spacer”.

However other known spacers may be used such as a polyglycol chain such as hexanediol, octanediol or polyethylene glycol (PEG).

In one embodiment, the donor molecule is attached at the 5′ end of the primer and the acceptor molecule is attached to the 3′ end of the probe.

In another embodiment, the acceptor molecule is attached to the 5′ end of the primer and the donor molecule is attached to the 3′ end of the probe.

Oligonucleotides of the system may be adapted for use in a trans dual hybridisation assay, for example,within such an assay in a multiplex arrangement.

In a particular embodiment, the said oligonucleotide primer is a forward or reverse primer for use in an amplification reaction, and the system further comprises a second oligonucleotide primer able to act as the reverse or forward primers respectively, in the amplification of said target sequence.

In a particular embodiment, the system of the present invention comprises at least one of the interactive probe/primer pairs described in copending patent application No. PCT/GB2011/050508.

The system of the invention may be used in conjunction with a primer extension reaction that yields a labelled primer extension product, optionally in the context of an amplification reaction such as polymerase chain reaction. This amplified product can then be probed using the oligonucleotide probe. A change in signal is indicative of an interaction between the first and second labels.

According to a further aspect of the invention there is provided a method for detecting the presence or amount of a target nucleic acid molecule comprising:
a) providing a sample containing or suspected of containing said nucleic acid molecule;
b) exposing the sample to a labelled oligonucleotide primer of a system as described above under conditions that enable annealing of said primer to any target nucleic acid molecule present in the sample, and extension of any annealed primer;
c) exposing any nucleic acid extension product produced in step b) to a labelled oligonucleotide probe of a system as described above under conditions that enable annealing of the probe to any complementary nucleic acid sequence in the extension product;
d) detecting an interaction between said first and second labels; and
e) relating this to the presence or amount of the target nucleic acid molecule in the sample.

The method can be carried out in the context of an ongoing amplification reaction such as a polymerase chain reaction. In such cases, the oligonucleotide probe is present throughout the reaction. In this case, continuous monitoring of the signal such as the fluorescent signal from the sample throughout the amplification reaction will allow the progress of any amplification reaction to be monitored. Thus, the amount of target nucleic acid molecule present in the sample may be quantified using conventional analytical methods.

Alternatively or additionally, the interaction may be monitored in the course of a melting or hybridisation assay, wherein the signal from the sample is monitored whilst the temperature is changed. The interaction of the first and second labels gives rise to a signal which will occur as the probe anneals to the primer extension product during a cooling operation. The signal will break down when the probe/primer extension product duplex destablises as the temperature rises. Melting analysis, carried out during or at the end of a primer extension or amplification reaction as described above, will allow further distinction/confirmation of the particular products obtained, for instance, in a multiplex reaction, where different products are obtained. Suitably different probes may be designed such that the annealing or destabilisation/melt temperatures are different. Therefore, a signal at a particular temperature will be characteristic of a particular target nucleic acid molecule or sequence within said molecule.

According to a further aspect of the invention, there is provided a kit for performing the above-mentioned method of the invention, comprising a system as described above and at least one reagent necessary to carry out a primer extension reaction. Such reagents may be selected from one or more of a polymerase enzyme, nucleotides, salts such as magnesium or manganese salts and buffers, in particular, PCR buffers.

Suitably, where a polymerase enzyme is present, it is an enzyme that has reduced or lacks 5′-3′ exonuclease activity so that it does not digest any annealed probe during the course of a primer extension reaction.

According to a further aspect of the invention there is provided, in a multiplex assay, the use of a system according to the invention.

According to a further aspect of the invention there is provided, in a multiplex assay, the use of a method for detecting a target sequence within a target region of a nucleic acid molecule according to the invention.

In one embodiment, the target nucleic acid molecule contains a target sequence in the form of a non-synonymous mutation or polymorphism that has phenotypic consequences of interest. For example, the system of the invention may be designed to identify a particular mutation that is responsible for a particular disease condition or the conferring of a particular characteristic, such as resistance to a particular drug or toxin. A particular example of interest is the identification of drug resistance in certain microorganisms and, particularly, viruses which are highly adaptive and so highly mutagenic/polymorphic. To this end, the probe to be used with the primer of the invention is designed to be specific for the mutation of interest.

One exemplary use of the invention is in the field of influenza diagnosis. Influenza viruses are RNA viruses and the most common type of flu virus is Influenza A. Within Influenza A there are several serotypes categorised on the basis of antibody responses to them, of which the most well known are H5N1 (avian flu) and H1N1 (swine flu). The “H” denotes hemagglutinin and the “N” neuraminidase, both proteins expressed on the surface of the flu virus and which exhibit the variations which give rise to the different antibody responses to the different serotypes of the virus.

During the 2009 worldwide outbreak of H1N1 swine flu, the antiviral drug Tamiflu® was a key means of combating viral infection and inhibiting the spread of the virus. However, some strains of the virus were found to be resistant to Tamiflu®. Identification of individuals carrying such a strain was only possible when treatment with Tamiflu® had been found to be ineffective, at which stage alternative treatment using a drug such as Relenza® would be appropriate. It would, of course, have been preferable to be able to identify the presence of a resistant strain before treatment began, so as to provide effective treatment more quickly and also to reduce the risk of the further transmission of the Tamiflu® resistant strain.

Resistance to the Tamiflu® drug is most commonly present when the polymorphisms causing the amino acid changes H274Y and N294S are present in the neuraminidase gene in N1 subtypes of Influenza A. A screening method to identify the presence of these polymorphisms is therefore required, which can provide rapid results at a reasonable cost.

Accordingly, the invention may be practised to identify the H274Y Tamiflu® (Oseltamivir) resistance mutation located in the neuraminidase gene in influenza virus and particularly in influenza type A; of the HA sub-type H1 or H5; of the sub-type N1 i.e. H1N1 or H5N1.

However, the invention has application in the detection of target sequences of interest in animals and microbes, particularly humans, bacteria, other viruses and pathogenic organisms. Typically, but not exclusively, the invention has particular application in the detection of target sequences in highly adaptive or evolving organisms.

As mentioned above, a particular embodiment useful in detection of polymorphisms in a target sequence, for example, two polymorphisms positioned within about 20-30 nucleotides of one another, is that disclosed in co-pending application no. PCT/GB2011/050508. In this embodiment, the invention provides a method of detecting the presence in a sample of a first target sequence and a second target sequence within a test region of a nucleic acid sequence, comprising conducting a nucleic acid amplification reaction to form a forward amplicon strand and a reverse amplicon strand of the test region, contacting the forward amplicon strand with a first probe labelled with a first FRET label and capable of hybridising to the first target sequence or complement thereof in the forward amplicon strand, and contacting the reverse amplicon strand with a second probe labelled with a second FRET label and capable of hybridising to the second target sequence or complement thereof in the reverse amplicon strand; wherein the nucleic acid amplification reaction is conducted using a forward amplification primer labelled with a third FRET label and a reverse amplification primer labelled with a fourth FRET label, the forward primer being incorporated into the forward amplicon strand and the second primer being incorporated into the reverse amplicon strand during the amplification reaction; and further wherein the first and third FRET labels form a first FRET pair and the second and fourth FRET labels form a second FRET pair, each FRET pair comprising a donor label; the method further comprising the steps of exposing the sample to an excitation source having a wavelength which excites the donor label in the first FRET pair and the donor label in the second FRET pair, detecting fluorescence from the sample and relating this to the presence or absence of the first and second target sequences. The first, second, third and fourth FRET labels may be arranged on the primer/probe molecules (as applicable) as disclosed in the present application. In particular, the label on the first primer may be linked to the 5′ end of the primer oligonucleotide and the label on the second primer may be linked to the 5′ end of the primer oligonucleotide.

It will be apparent to those skilled in the art that the precise design of a primer or probe to detect a target sequence can be undertaken having regard to conventional techniques and the use of conventional programming tools. Knowledge of the sequence structure of a target region enables a complementary oligonucleotide primer or probe to be made. In this way the invention has application in the ways described above and, moreover, in the multiple species described above.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Particular features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF THE FIGURES

FIG. 1 shows a diagram of a trans dual hybridisation assay using the standard Universal Donor signalling arrangement (acceptor on the probe); and

FIG. 2 shows a diagram of the seminal trans dual hybridisation assay produced by other workers (Bernard et al. supra).

Embodiments of the invention will now be described, by way of example only, with reference to FIGS. 3-9 wherein:

FIG. 3 shows a diagram of a trans dual hybridisation assay using a system of the invention;

FIG. 4 shows a diagram of a trans dual hybridisation assay tested using an alternative embodiment of the system of the invention;

FIG. 5 shows a diagram of a trans dual hybridisation assay tested using a further alternative embodiment of the system of the invention;

FIG. 6 shows a series of comparative graphs showing the results of an assay in accordance with FIG. 1 compared with that of FIG. 3: a. H5N1 TRANS-DH PoP assay with an internal and a 5′labelled 6FAM donor primer. Detection of 10⁴+10³ copies/rxn H5N1 WT DNA by real-time PCR and melt analysis, 530 nm 6FAM donor response; b. H5N1 TRANS-DH PoP assay, internal vs. 5′ end labelled 6FAM donor primer. Detection of 10⁴+10³ copies/rxn H5N1 WT DNA by real-time PCR and melt analysis. 670 nm TYE665 probe response; c. H5N1 TRANS-DH PoP assay, internal vs. 5′ end labelled 6FAM donor primer. Detection of 10⁴+10³ copies/rxn H5N1 SEQ ID NO:1 DNA by real-time PCR and melt analysis, 530 nm 6FAM donor response; d. H5N1 TRANS-DH PoP assay, internal vs. 5′ end labelled 6FAM donor primer. Detection of 10⁴+10³ copies/rxn H5N1 SEQ ID NO:1 DNA by real-time PCR and melt analysis, 670 nm TYE665 probe response; e. H5N1 TRANS-DH PoP assay, internal vs. 5′ end labelled 6FAM donor primer. Detection of 10⁴+10³ copies/rxn H5N1 SEQ ID NO:2 DNA by real-time PCR and melt analysis, 530 nm 6FAM donor response; f. H5N1 TRANS-DH PoP assay, internal vs. 5′ end labelled 6FAM donor primer. Detection of 10⁴+10³ copies/rxn H5N1 SEQ ID NO:2 DNA by real-time PCR and melt analysis, 670 nm TYE665 probe response (with reference to Table 7, curves 1-4=Mix lB H5N1 WT DNA, 5-8=Mix 1B H5N1 SEQ1 DNA, 9-12=Mix 1B H5N1 SEQ2 DNA, 13 & 14=Mix 1B H5N1 NTC, 15-18=Mix 1C H5N1 WT DNA, 19-22=Mix 1C SEQ1 DNA, 23-26=Mix 1C H5N1 SEQ2 DNA, 27 & 28=Mix 1C H5N1 NTC);

FIG. 7 is a series of graphs showing the results of an assay in accordance with FIG. 4; a. H5N1 TRANS-DH PoP assay, 5′ end labelled 6FAM donor primer with molecular spacer. Detection of 10⁴+10³ copies/rxn H5N1 WT DNA by real-time PCR and melt analysis, 530 nm 6FAM donor response; b. H5N1 TRANS-DH PoP assay, 5′ end labelled 6FAM donor primer with molecular spacer. Detection of 10⁴+10³ copies/rxn H5N1 WT DNA by real-time PCR and melt analysis, 670 nm TYE665 probe response; c. H5N1 TRANS-DH PoP assay, 5′ end labelled 6FAM donor primer with molecular spacer. Detection of 10⁴+10³ copies/rxn H5N1 SEQ ID NO:1 DNA by real-time PCR and melt analysis, 530 nm 6FAM donor response; d. H5N1 TRANS-DH PoP assay, 5′ end labelled 6FAM donor primer with molecular spacer. Detection of 10⁴+10³ copies/rxn H5N1 SEQ ID NO:1 DNA by real-time PCR and melt analysis, 670 nm TYE665 probe response; e. H5N1 TRANS-DH PoP assay, 5′ end labelled 6FAM donor primer with molecular spacer. Detection of 10⁴+10³ copies/rxn H5N1 SEQ ID NO:2 DNA by real-time PCR and melt analysis, 530 nm 6FAM donor response; f. H5N1 TRANS-DH PoP assay, 5′ end labelled 6FAM donor primer with molecular spacer. Detection of 10⁴+10³ copies/rxn H5N1 SEQ ID NO:2 DNA by real-time PCR and melt analysis, 670 nm TYE665 probe response (curves 1-4=Mix 2B H5N1 WT DNA, 5-8=Mix 2B H5N1 SEQ1 DNA, 9-12=Mix 2B H5N1 SEQ2 DNA, 13 & 14=Mix 2B H5N1 NTC, 29=Mix lB WT control, 30=Mix lB SEQ1 control, 31=Mix 1B SEQ2 control);

FIG. 8 is a series of graphs showing the results of an assay in accordance with FIG. 5; a. H5N1 TRANS-DH PoP assay, 5′ end labelled TYE665 reporter-labelled primer. Detection of 10⁴+10³ copies/rxn H5N1 WT DNA by real-time PCR and melt analysis, 530 nm 6FAM donor probe response; b. H5N1 TRANS-DH PoP assay, 5′ end labelled TYE665 reporter-labelled primer. Detection of 10⁴+10³ copies/rxn H5N1 WT DNA by real-time PCR and melt analysis, 670 nm TYE665 primer response; c. H5N1 TRANS-DH PoP assay, 5′ end labelled TYE665 reporter-labelled primer. Detection of 10⁴+10³ copies/rxn H5N1 SEQ ID NO:1 DNA by real-time PCR and melt analysis, 530 nm 6FAM donor probe response; d. H5N1 TRANS-DH PoP assay, 5′ end labelled TYE665 reporter-labelled primer. Detection of 10⁴+10³ copies/rxn H5N1 SEQ ID NO:1 DNA by real-time PCR and melt analysis, 670 nm TYE665 primer response; e. H5N1 TRANS-DH PoP assay, 5′ end labelled TYE665 reporter-labelled primer. Detection of 10⁴+10³ copies/rxn H5N1 SEQ ID NO:2 DNA by real-time PCR and melt analysis. 530 nm 6FAM donor probe response; f. H5N1 TRANS-DH PoP assay, 5′ end labelled TYE665 reporter-labelled primer. Detection of 10⁴+10³ copies/rxn H5N1 SEQ ID NO:2 DNA by real-time PCR and melt analysis, 670 nm TYE665 primer response; g. H5N1 PoP assay vs. SEQ ID NO:1 DNA, comparison of donor quenching PCR profiles (530 nm) exemplifying the cosmetic improvement in FRET-mediated 6FAM quenching from reversing the primer-probe label configuration; h. H5N1 PoP assay vs. SEQ ID NO:1 DNA, comparison of donor quenching melt analysis profiles (530 nm) exemplifying the reduction in proximal quenching within the amplification product signalling (curves 15-18=Mix 2C H5N1 WT DNA, 19-22=Mix 2C H5N1 SEQ1 DNA, 23-26=Mix 2C H5N1 SEQ2 DNA, 27 & 28=Mix 2C H5N1 NTC, 29=Mix 1B WT control, 30=Mix 1B SEQ1 control, 31=Mix lB SEQ2 control); and

FIG. 9 shows the consensus sequence derived from the neuraminidase gene of influenza A (SEQ ID NO:4) with positions donated “−” representing sequence degeneracy or sites of insertion.

### EXAMPLES

Assay Design

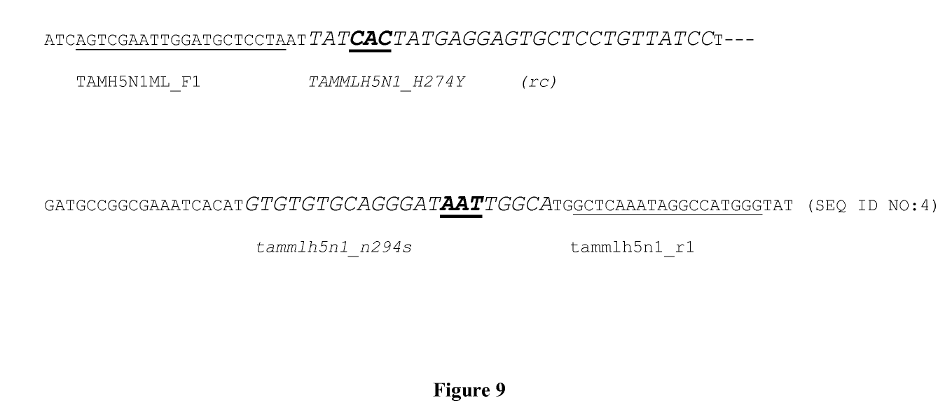

Assay design and development was conducted using a super-consensus derived from H1N1 and H5N1 sequences which is shown in FIG. 9. In FIG. 9, the underlined sequences represent the forward and reverse primer regions. The probe regions are labelled in italics. The underlined codon “CAC” (within region bound by the probe TAMMLH5N1_H274Y) is that encoding the amino acid Histamine at position 274 in the neuraminidase protein. Alteration of this to TAT or TAC results in expression of Tyrosine at this position. The underlined codon “AAT” (within the region bound by the probe TAMMLH5N1_N294S) is that encoding the amino acid Asparagine at position 294. Alteration of this from AAT to TCT, TCC, TCA or TCG results in expression of Serine at this position.

The constructs below were synthesised and used as model templates to exemplify the method. “(rc)” in FIG. 9 denotes that the reverse complement oligonucleotide was used in the described experiments.

Wild Type Sequence (SEQ ID NO:3):

ATCAGTCGAATTGGATGCTCCTAATTAT [symbol] TATGAGGAGTGCTCCTGT
TATCCTTTTGATGCCGGCGAAATCACATGTGTGTGCAGGGAT [symbol] TGGC
ATGGCTCAAATAGGCCATGGGTAT

SEQ ID NO:1: The following sequence has one mismatch in the forward primer binding region (underlined), one in the reverse primer binding region, and one in the H274Y probe binding region near the mutation site (marked in lower case).

ATCAGTaGAATTGGATGCTCCTAATTAc [symbol] TATGAGGAGTGCTCCTGT
TATCCTGATGCCGGCGAAATtACATGTGTGTGCAGGGAT [symbol] TGGCATG
GtTCAAATAGGCCATGGGTAT

SEQ ID NO:2: The following sequence has one mismatch in the forward primer binding region, one in the reverse primer binding region, and one in the H274Y probe binding region away the mutation site (marked in lower case).

ATCAGTCGAATTGGATGCTCCcAATTAT [symbol] TATGAGGAaTGCTCCTGT
TATCCTGATGCCGGCGAAATCACATGTGTGTGCAGGGAT [symbol] TGGCATG
GCTCAAATcGGCCATGGGTAT

This example represents an H5N1 specific assay. This resistance marker assay utilises the dye (6FAM) as the donor label and either TYE665 or TYE700 as the acceptor moiety.

A key to achieving a high specificity for the resistance status is the generation of robust melting peak motifs for analysis by an automated algorithm. This will occur in a multiplex reaction achieved using different fluorophores whose emissions are measured as part of a Universal Donor Fluorescent Resonant Energy Transfer (FRET) system.

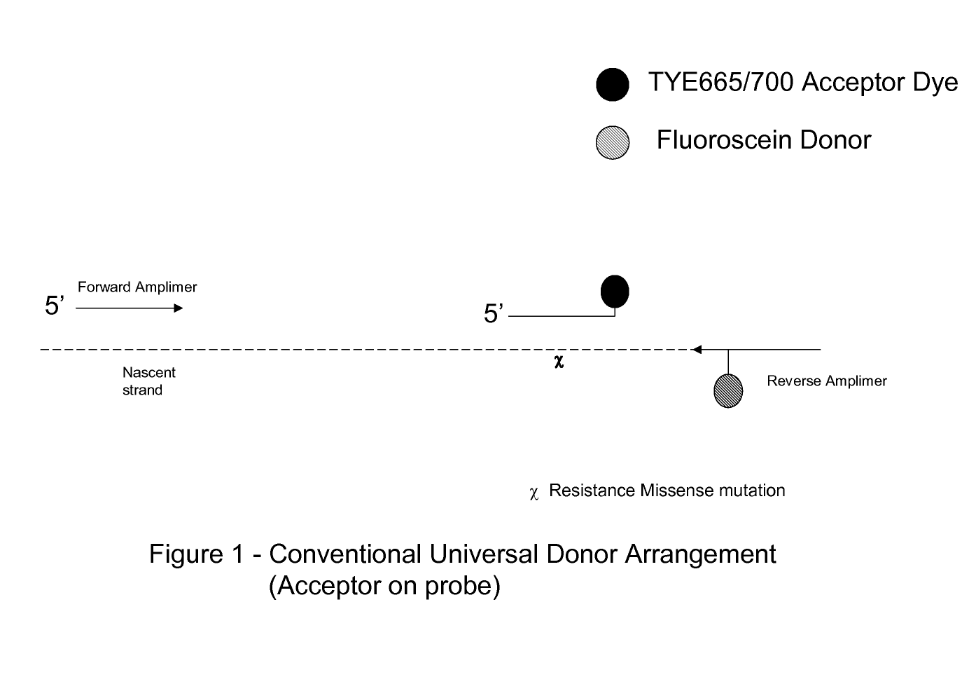

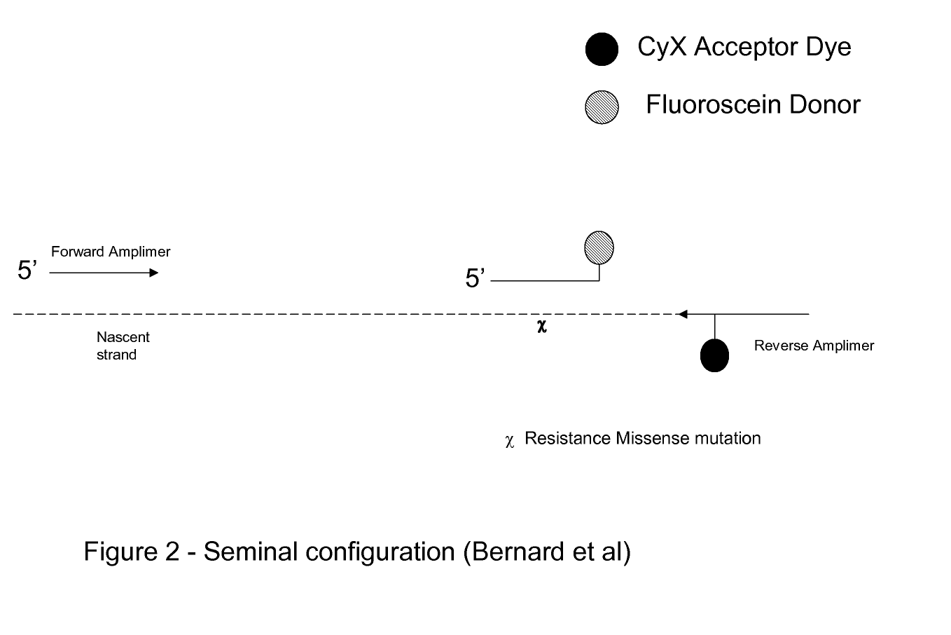

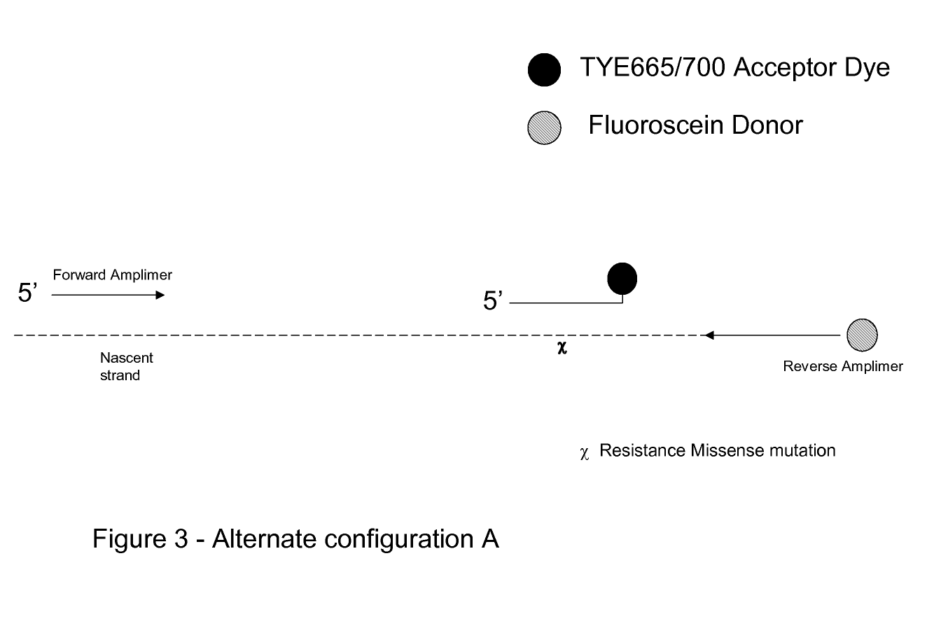

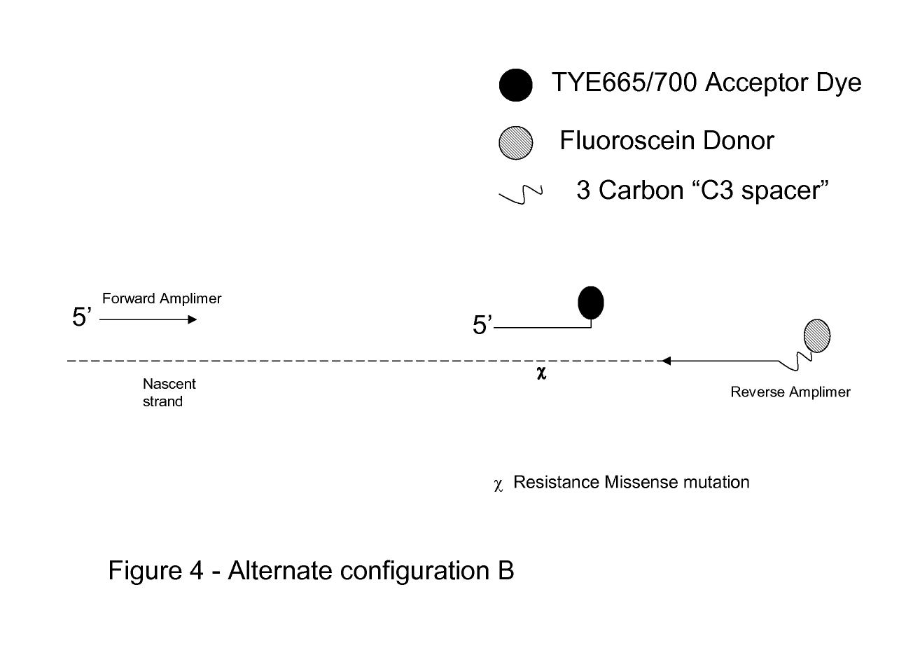

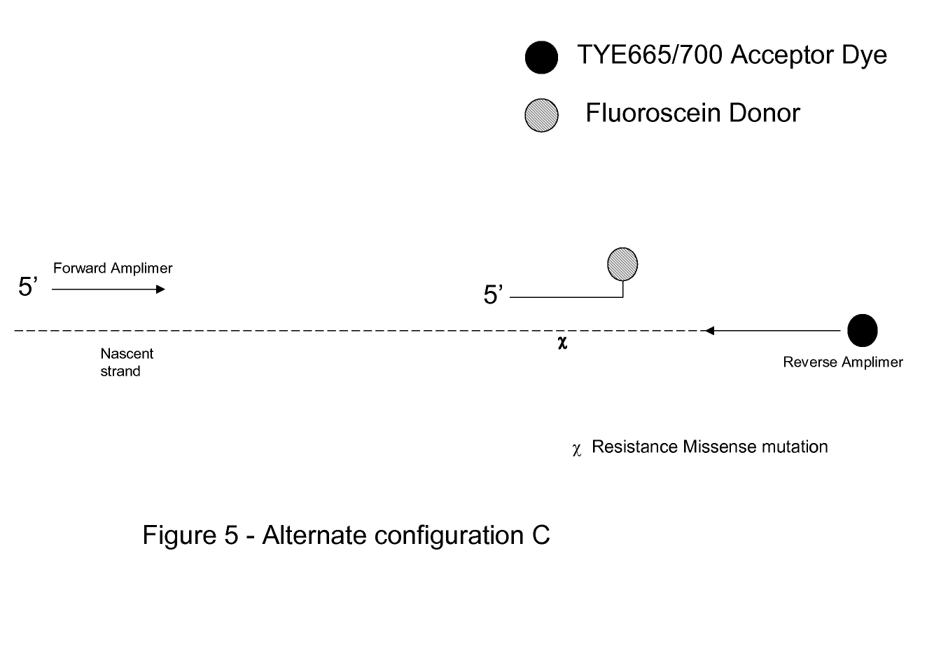

In the following examples three assay designs were tested.
a. Method 1. Place the 6FAM at the 5′ end of the primer. This distances the 6-FAM from interaction with the amplimer sequence (FIG. 3). This was directly compared with a conventional trans Dual hybridisation assay using an internally labelled primer (FIG. 1).
b. Method 2. Place the 6FAM at the 5′ end of the primer but at the end of a spacer. This distances the 6FAM from interaction with the amplimer sequence whilst also providing greater opportunity for Dexter quenching of the FRET pair (FIG. 4).
c. Method 3. Switch the dye configuration such that the Reporter (acceptor) TYE™ dyes are located at the 5′ end of the primer and place the donor at the 3′ of the probe (FIG. 5).

Materials and Methods
1.1 Equipment
   Qiagility Liquid Handling System
   Roche LightCycler® 2.0 (EDLE213)
1.2 Materials
   20 μl LC glass capillaries
   5 ml Corbett Diluent Bijou tubes
   1.5 ml Eppendorf Tubes
   50 μl+200 μl robot tips
   200 μl Microamp tubes
1.3 Reagents
   RNase-DNase free water
   500 mM Tris-HCl pH8.8
   20 mg/ml Non-acetylated BSA
   100 mM MgCl₂
   2 mM DUTPs
   5 U/μl Taq DNA Polymerase
   10 μM TAMH5N1ML_R1
   10 μM TAMH5N1ML_F1
   10 μM TAMMLH5N1_H274Y TYE665
   10 μM TAMH5N1ML_F1 5′ F
   10 μM TAMH5N1ML_F1 5′SP
   10 μM TAMH5N1ML_F1 5′TYE665
   10 μM TAMMLH5N1_H274Y 6FAM
   H5N1 ‘WT’ Plasmid DNA template
   H5N1 ‘SEQ ID NO 1’ Long-oligo DNA template
   H5N1 ‘SEQ ID NO 2’ Long-oligo DNA template

The primers and probes used had the sequences shown in Table 1:

TABLE 1

| Primer/ | | | SEQ |
|---|---|---|---|
| probe | Name | Sequence 5′-3′ | ID NO |
| Forward | TAMH5N1ML_F1 | AGTCGAATTGGATGCTCCTA | 5 |
| primer | | | |
| Reverse | TAMH5N1ML_R1 | CCCATGGCCTATTTGAGC | 6 |
| primer | | | |
| Probe | TAMMLH5N1_H274Y | GGATAACAGGAGCACTCCTC | 7 |
| | | ATAGTGATA | |

The primers and probes utilised the following labelling positions:

Unlabelled R Primer (TAMH5N1ML_R1)

Internal dT-labelled fluorescein F primer (TAMH5N1ML_F1)

3′ TYE665 end-labelled probe (TAMMLH5N1_H274Y TYE665)

5′ Fluorescein end-labelled primer (TAMH5N1ML_F1 5′)

5′ Fluorescein end-labelled primer with spacer (TAMH5N1ML_F1 5′SP)

5′ TYE665 end-labelled primer (TAMH5N1ML_F1 5′TYE665)

3′ Fluorescein end-labelled probe (TAMMLH5N1_H274Y 6FAM)

The cDNA sequence of FIG. 9 corresponds to a consensus sequence for a portion of the RNA sequence from all known strains of H5N1 influenza viruses. This part of the sequence includes the codons which, when altered, result in the H274Y and N294S mutations in the neuraminidase protein.

Suitable primers and hybridisation probes were identified, by methods routinely used by the skilled person (use of the open-source software JALVIEW in combination with the EMBL search toolset), as being suitable for amplification of regions of the above sequences which encompassed the two polymorphic sites. Probes for the N294S polymorphism were developed so as to be complementary to the reverse amplicon strand.

The primers and probes were labelled with either 6FAM or TYE655 at the 5′ and 3′ ends respectively using conventional methodology by Integrated DNA Techologies (IDT).

Polymerase chain reactions were carried out using the above sets of primers and probes and, at the conclusion of the PCR, a melt analysis was carried out for each sample.

The reagents used are set out in Table 2:

TABLE 2
components of PCR reaction mixtures

| | | | Volume per |
|---|---|---|---|
| | | | 20 μl Reaction |
| Reagent | Stock Conc | Final Conc | (μl) |
| Tris-HCl pH8.8 | 500 mM | 50 mM | 2 |
| BSA | 20 mg/ml | 0.25 μg/μl | 0.25 |
| MgCl2 | 100 mM | 3 mM | 0.6 |
| dUTPs | 2 mM | 0.2 mM | 2 |
| Forward Primer | 10 μM | 0.5 μM | 1 |
| Reverse primer | 10 μM | 0.5 μM | 1 |
| TYE665-labelled probe | 10 μM | 0.2 μM | 0.4 |
| TYE705-labelled probe | 10 uM | 0.2 μM | 0.4 |
| Anti-Taq Polymerase | 5 U/μl | 0.08 U/μl | 0.32 |
| Antibody | | | |
| Taq Polymerase | 5 U/μl | 0.04 U/μl | 0.16 |
| Template plasmid | — | | 2 |
| Nuclease-free H2O | — | — | 9.87 |

Table 3 shows the PCR temperature cycling conditions:

TABLE 3
PCR and melt analysis conditions

| Phase | | | | Target | Hold | Transition | Florescence | |
|---|---|---|---|---|---|---|---|---|
| | | No of | Segment | Temp | Time | Rate | Acquisition | |
| Number | Type | Cycles | Number | ° C. | s | ° C./s | Type | Channels |
| 1 | HOLD | 1 | 1 | 95 | 30 | 20 | — | — |
| 2 | Amplify | 50 | 1 | 95 | 5 | 20 | — | — |
| | | | 2 | 55 | 20 | 20 | Single | ALL |
| | | | 3 | 74 | 5 | 20 | — | — |
| 3 | MELT | 1 | 1 | 40 | 15 | 20 | — | — |
| | | | 2 | 95 | 0 | 0.1 | Continuous | ALL |

Results

Evaluation of the signalling modifications was completed over five LC2.0 experimental runs. See Table 7 below for a summary table of all experimental data. Experiments can be broken down into the following subsets.

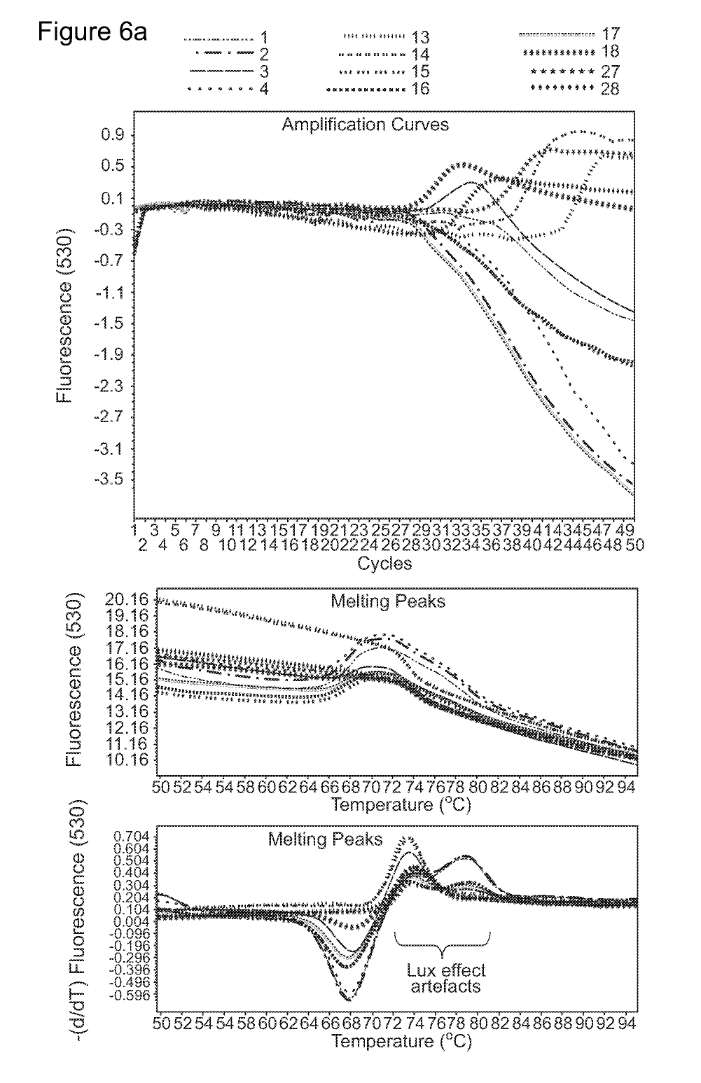

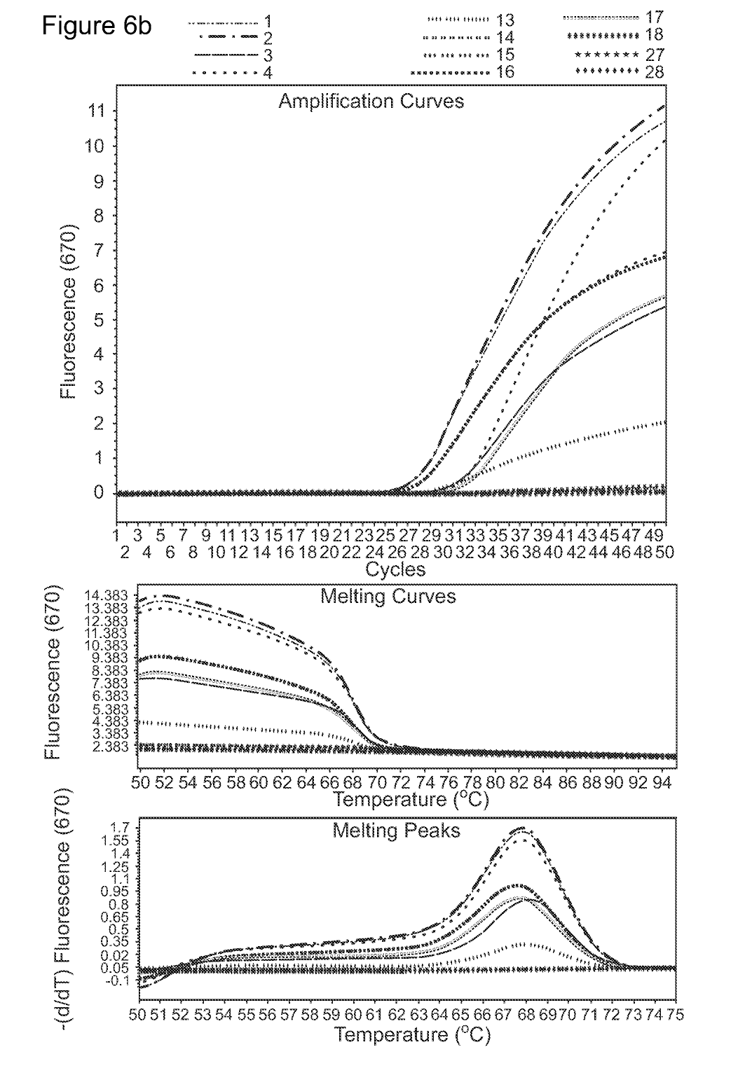

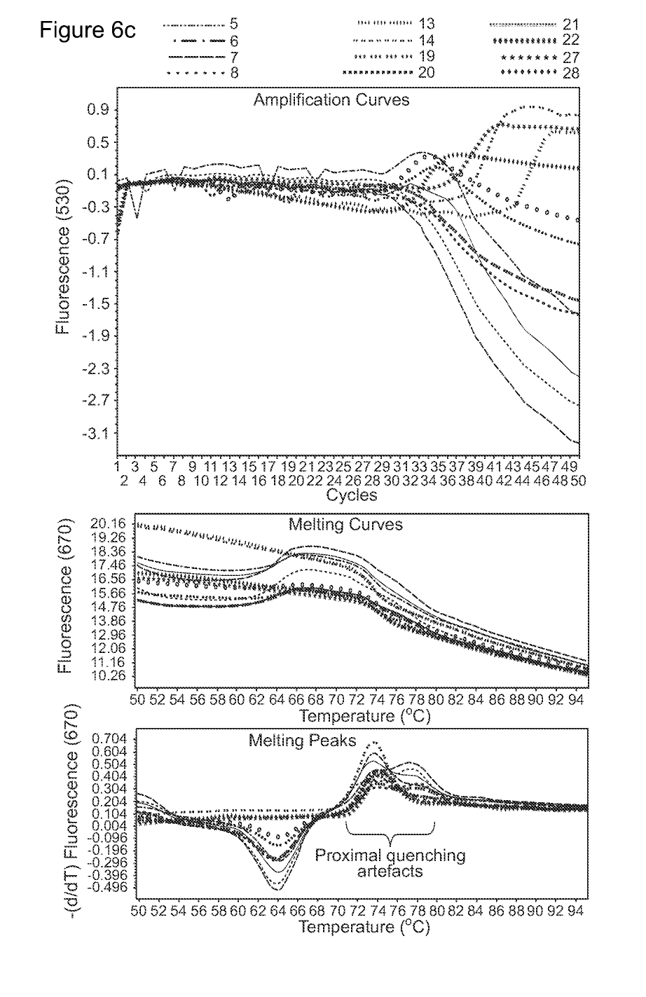

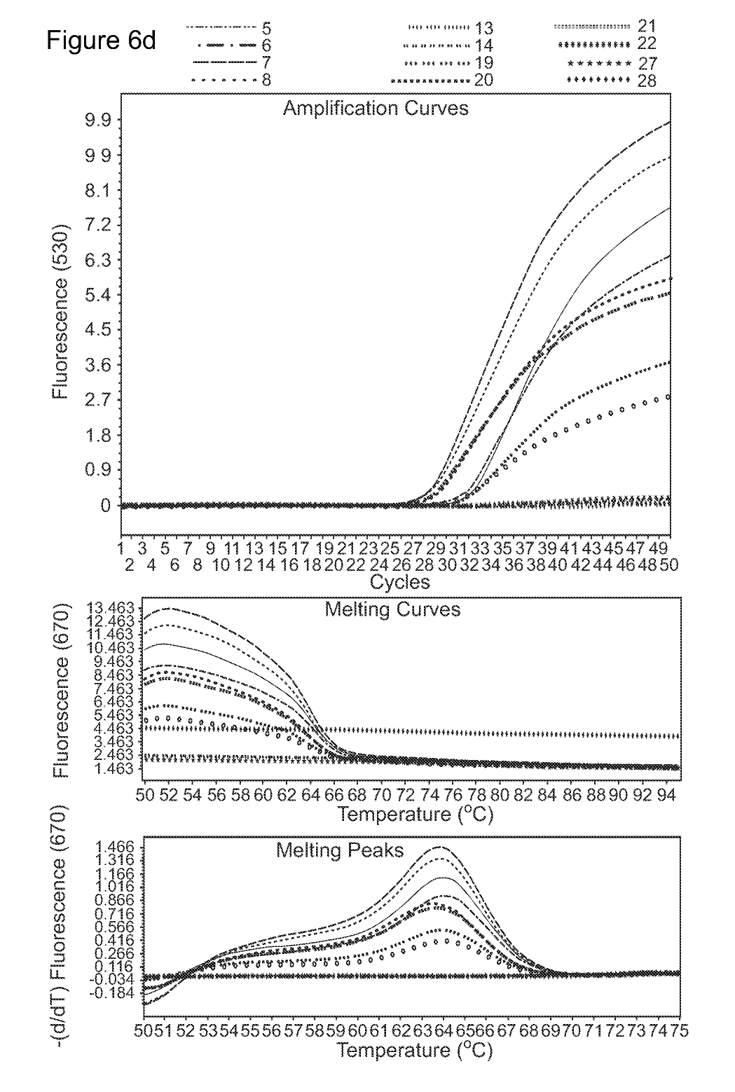

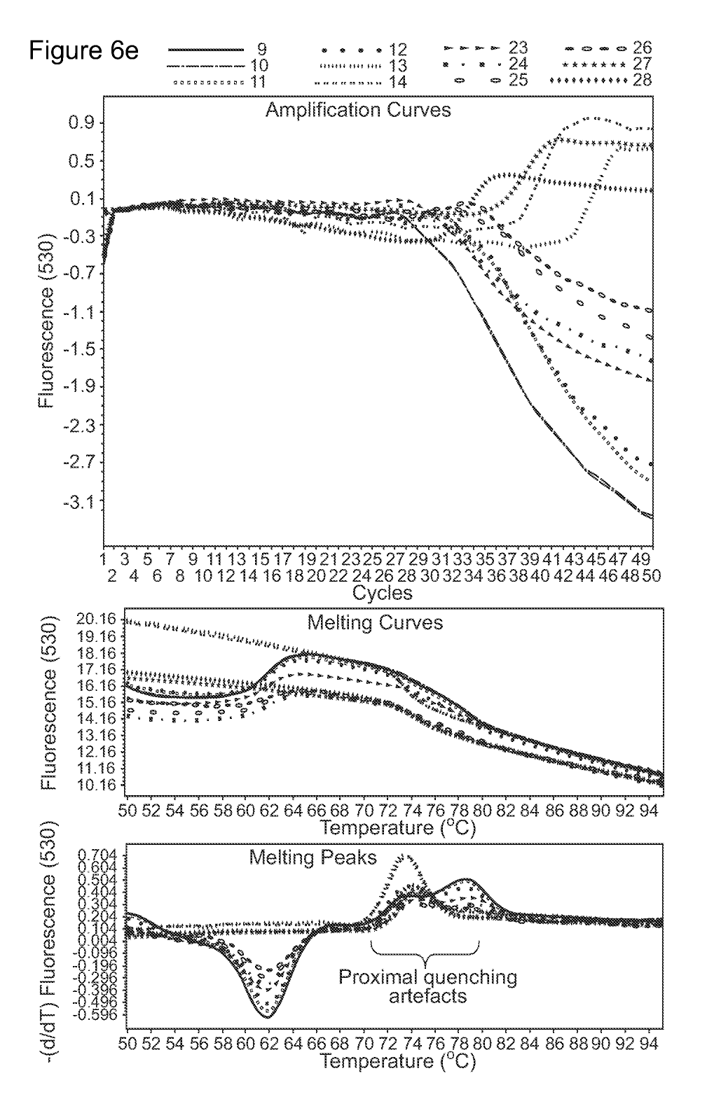

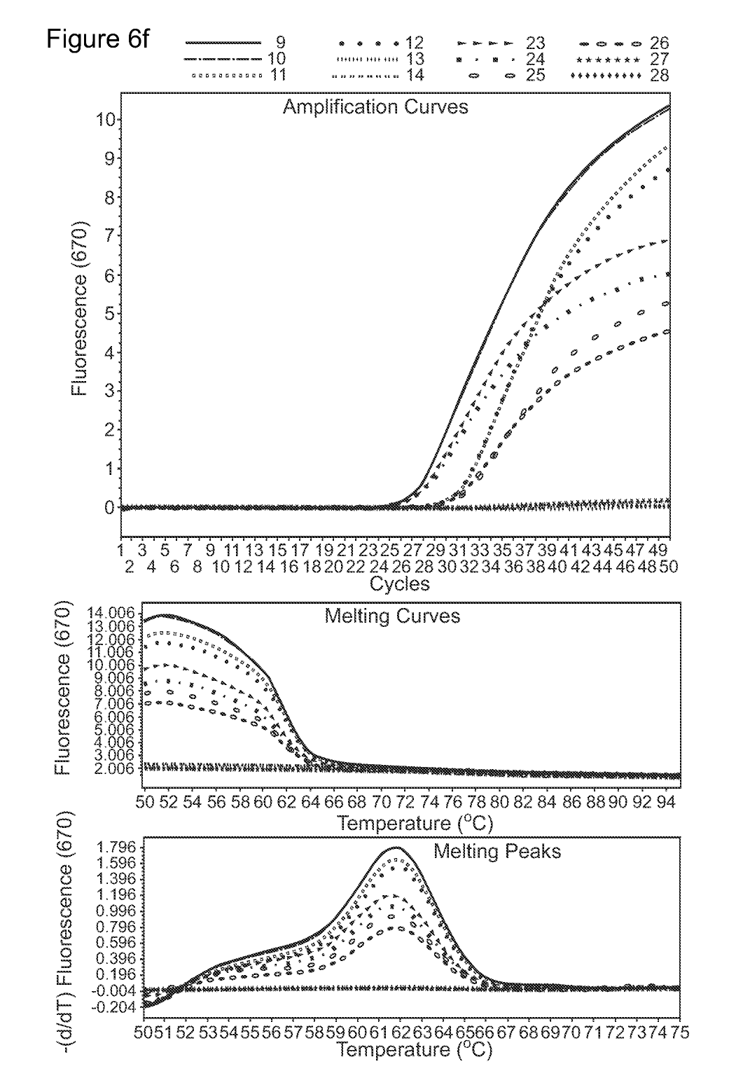

Method 1: Labelling Position (internal vs 5′ end 6FAM-labelled primer comparison) The conventional trans dual-hybridisation assay was directly compared with an equivalent assay using a 5′ 6FAM end-labelled primer to assess the criticality of donor labelling position on assay signalling (FIGS. 6a-f). The 5′ labelled primer generated a lower signal yield than observed for the conventional assay for all template types (WT, SEQ ID NO:1 and SEQ ID NO:2 DNA). See table 4. However, the signal was still clearly detectable and the assay had the advantage of using easy to prepare probes.

TABLE 4
comparative signal yield between internal vs. 5′ end labelled 6FAM donor

| | % 670 nm signal yield vs. |
|---|---|
| Sequence Type | internal 6FAM label |
| WT DNA | ≈−40% |
| SEQ ID NO 1 | ≈−30% |
| SEQ ID NO 2 | ≈−40% |

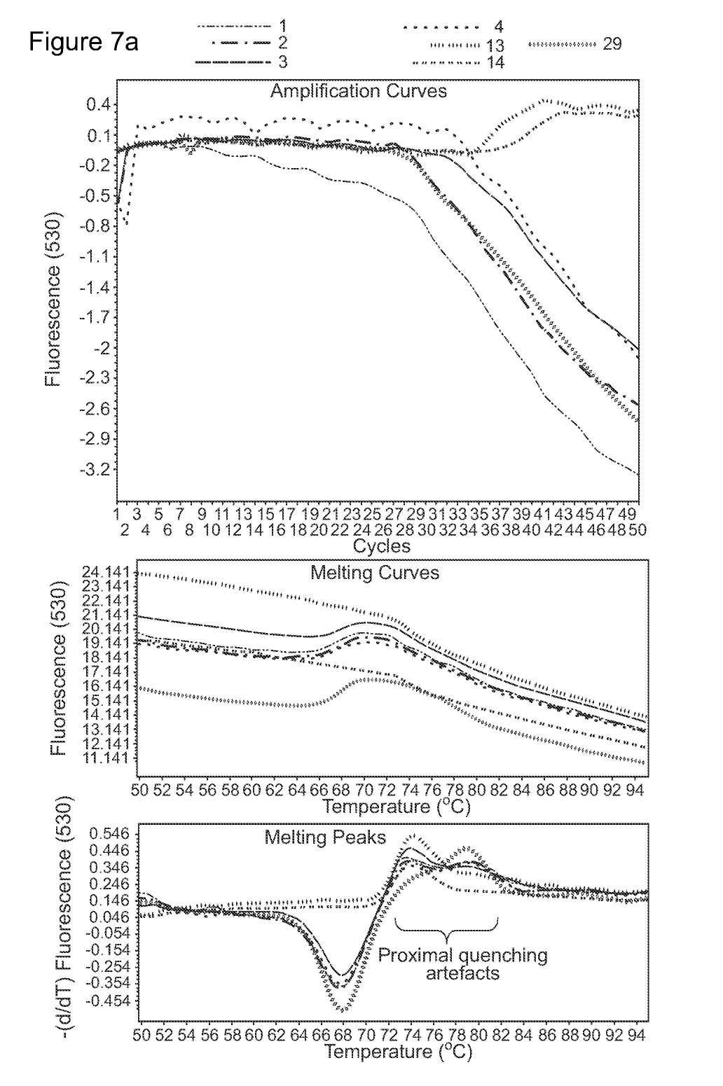

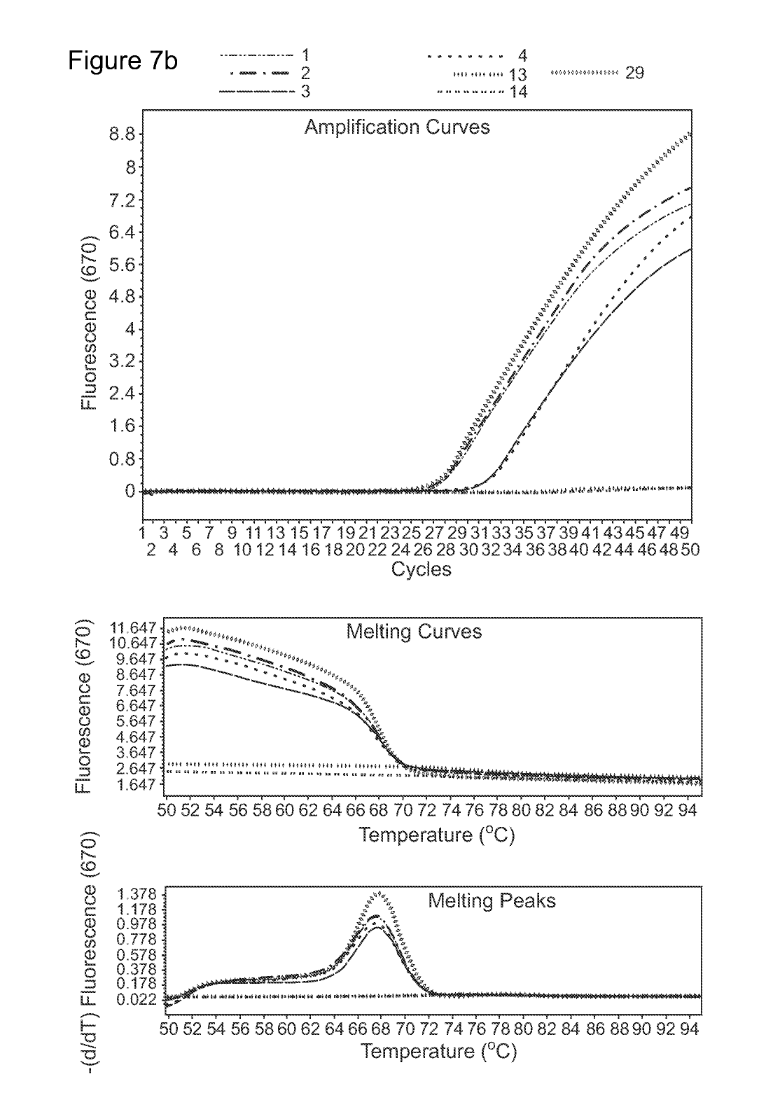

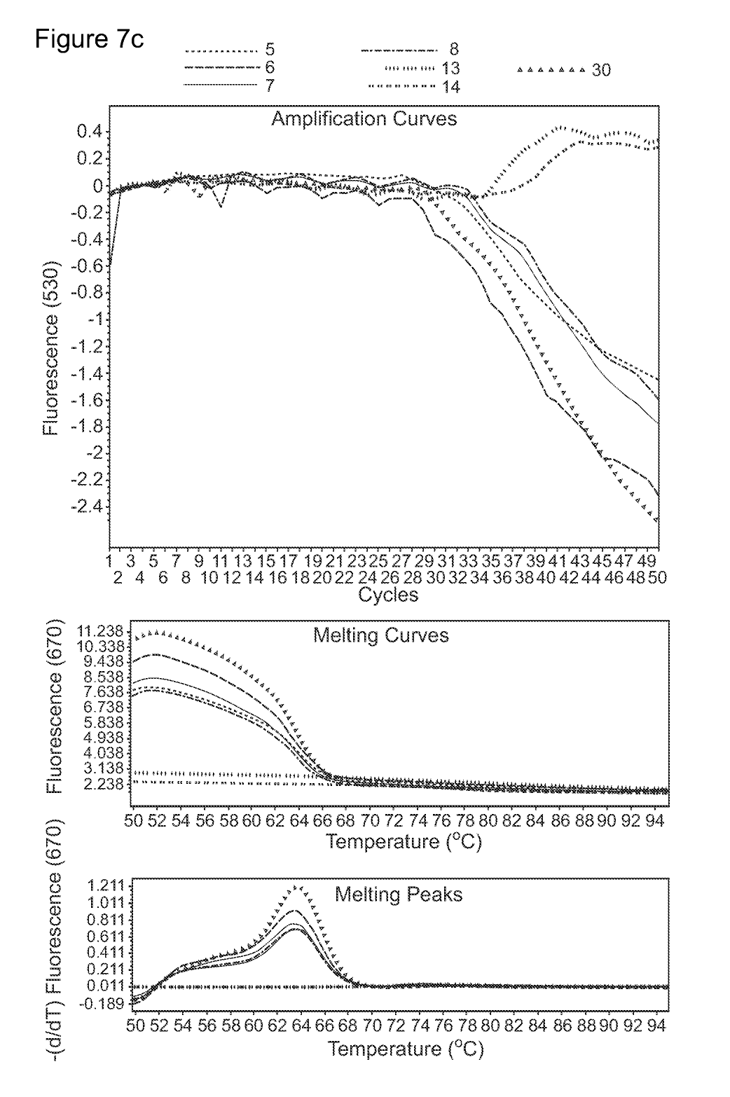

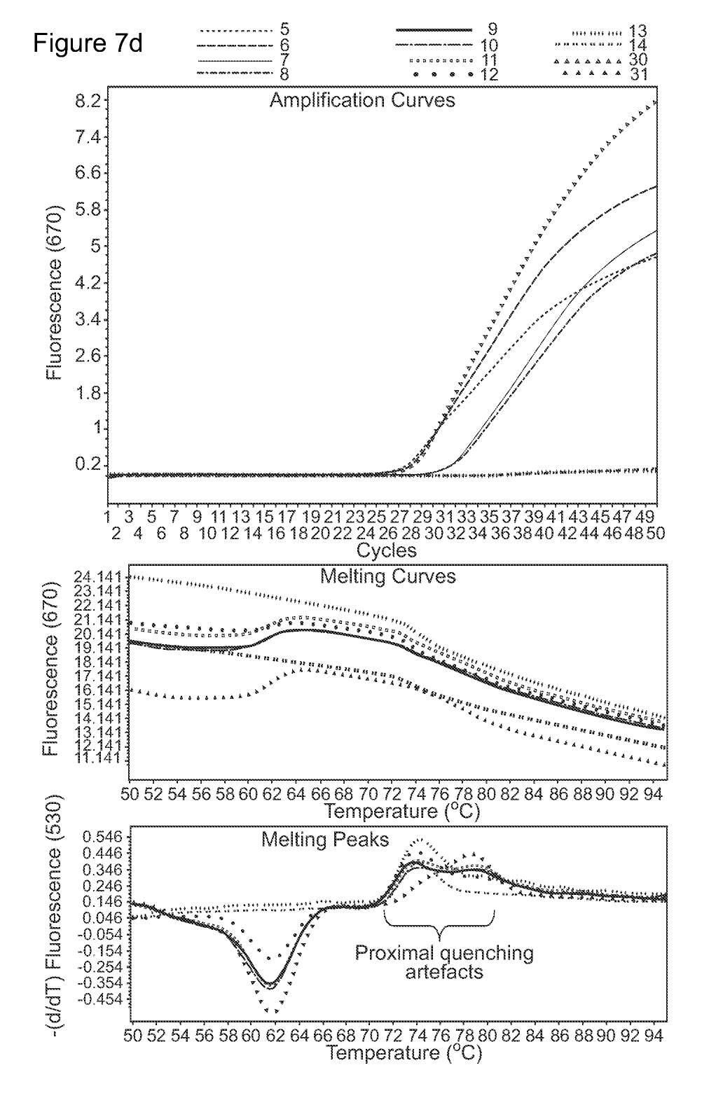

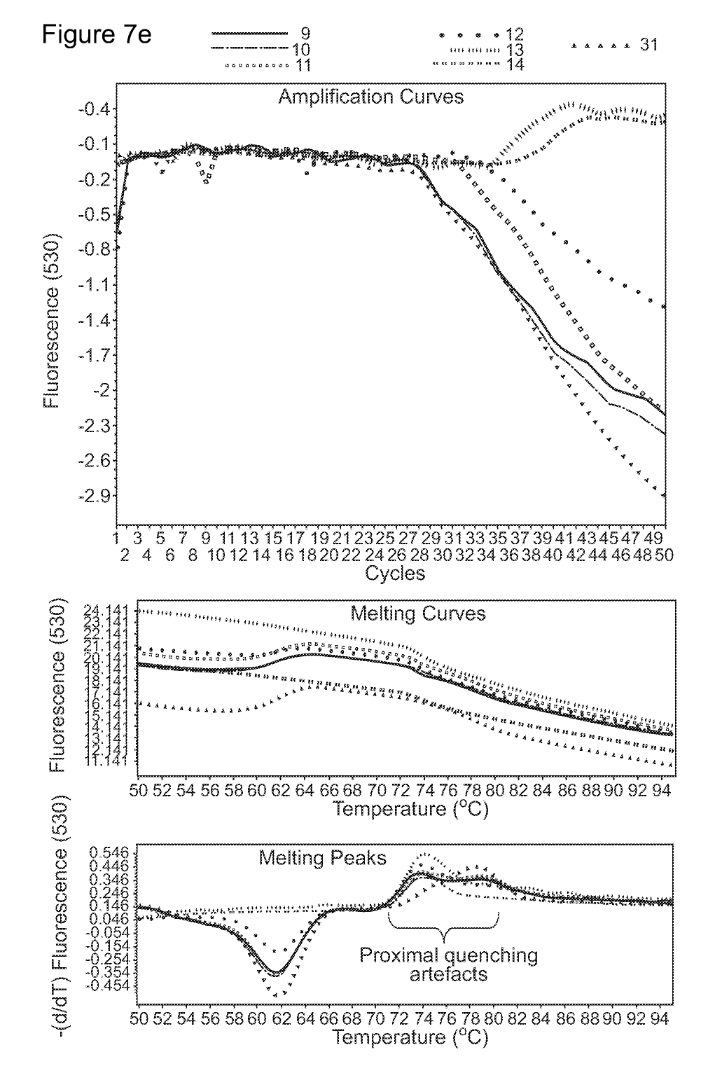

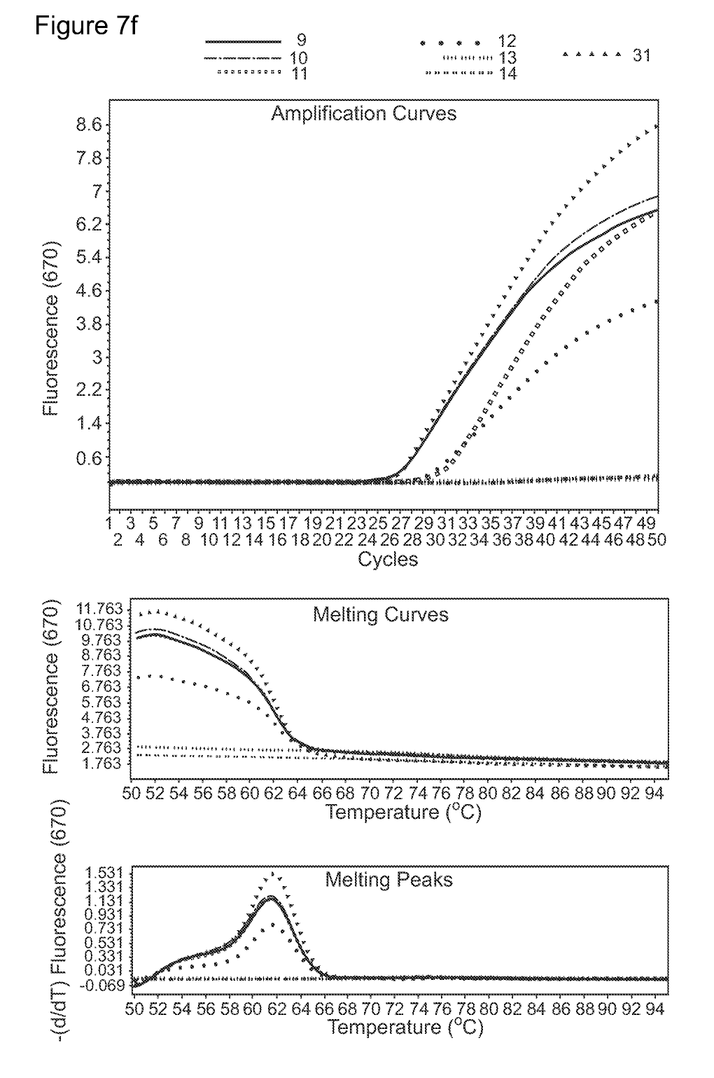

Method 2: Labelling Position (5′ end-labelled 6FAM-primer with molecular spacer) The conventional trans dual-hybridisation assay was directly compared with an equivalent assay using a 5′ 6FAM end-labelled primer combined with a C3 spacer to assess the criticality of donor labelling position on assay signalling. The results are shown in FIG. 7. 5′ 6FAM C3 spacer exhibited a reduced FRET compared to the internally labelled 6FAM spacer. However, this reduction was not as significant a reduction as observed for method 1. See Table 5.

TABLE 5
comparative signal yield between internal vs. 5′ 6FAM end-labelled
donor vs. 5′ 6FAM end-labelled + spacer donor.

| | % signal yield vs. | % 670 nm signal yield vs. |
|---|---|---|
| Sequence Type | 5′ end label | internal 6FAM label |
| WT DNA | ≈+3% | ≈−15% |
| SEQ ID NO 1 | ≈+8% | ≈−25% |
| SEQ ID NO 2 | ≈+0% | ≈−20% |

The unwanted proximal quenching was also reduced over that observed in the controls and/or Method 1, as is clear from FIG. 7.

Method 3: Probe label switch (5′ TYE665-labelled primer+3′ 6FAM-labelled probe)

To reduce the impact of adverse 6FAM-mediated fluorescent amplicon signalling, the conventional assay was compared against an equivalent assay using a 5′ TYE665-labelled primer with a 3′ 6FAM labelled probe (effectively reversing the donor and reporter signalling components). Compared with the standard donor primer-reporter probe configuration, signal yield from the TYE665-labelled primer embodiment was lower for all three DNA target types. See table 6.

TABLE 6
comparative TYE665 signal yield from a labelled reporter primer
and 6FAM-donor probe configuration vs. the standard
6FAM donor primer + TYE665 probe.

| | % 670 nm signal yield vs. standard |
|---|---|
| Sequence Type | internally-labelled 6FAM primer |
| WT DNA | ≈−30% |
| SEQ ID NO 1 | ≈−40% |
| SEQ ID NO 2 | ≈−35% |

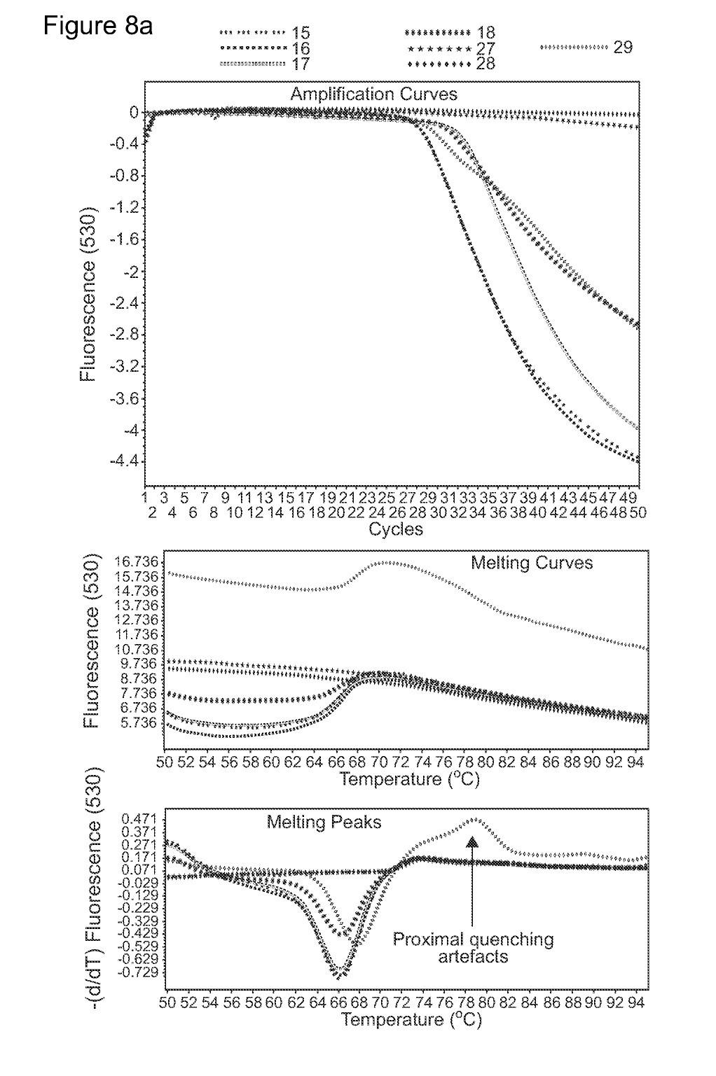

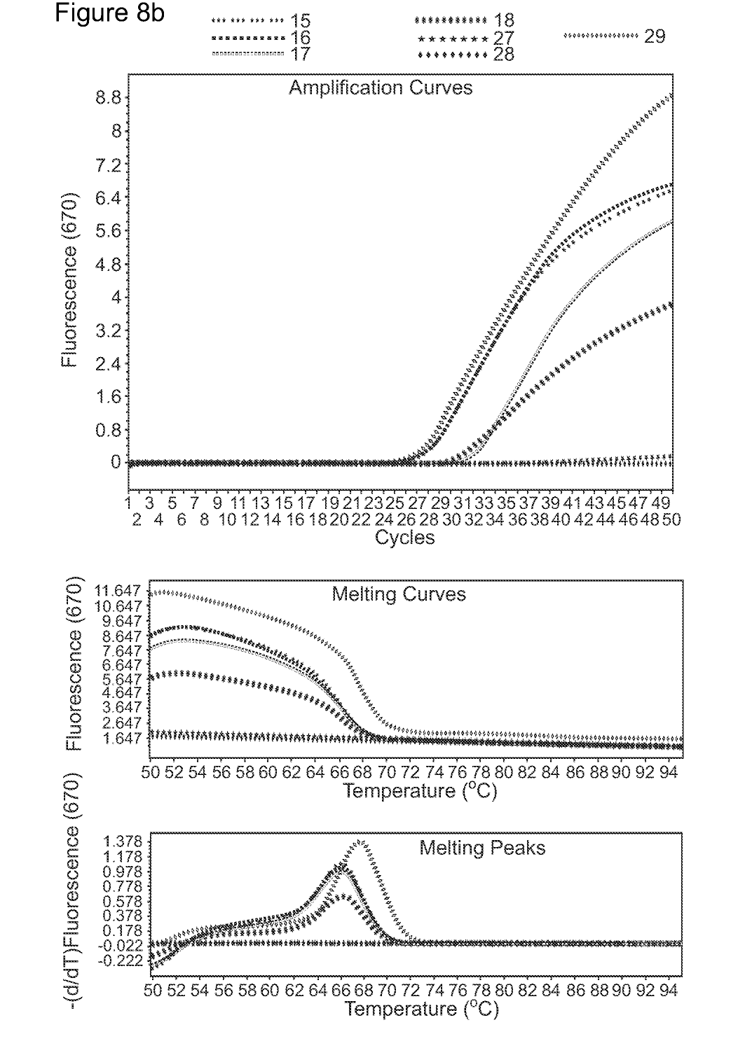

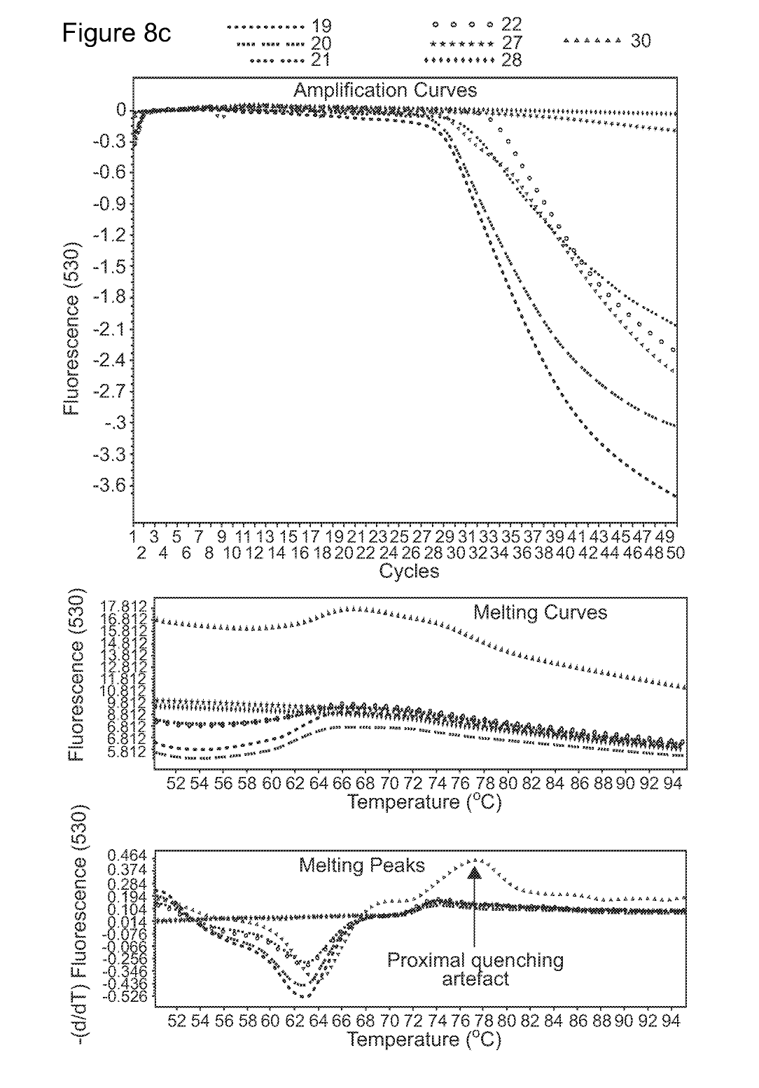

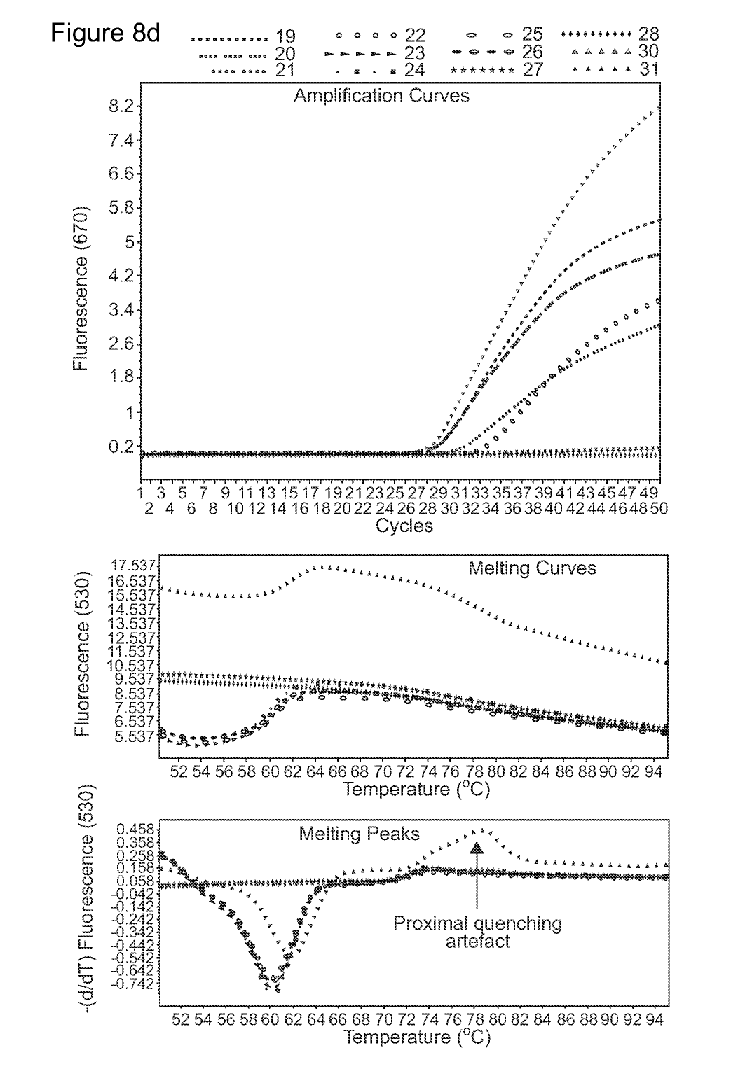

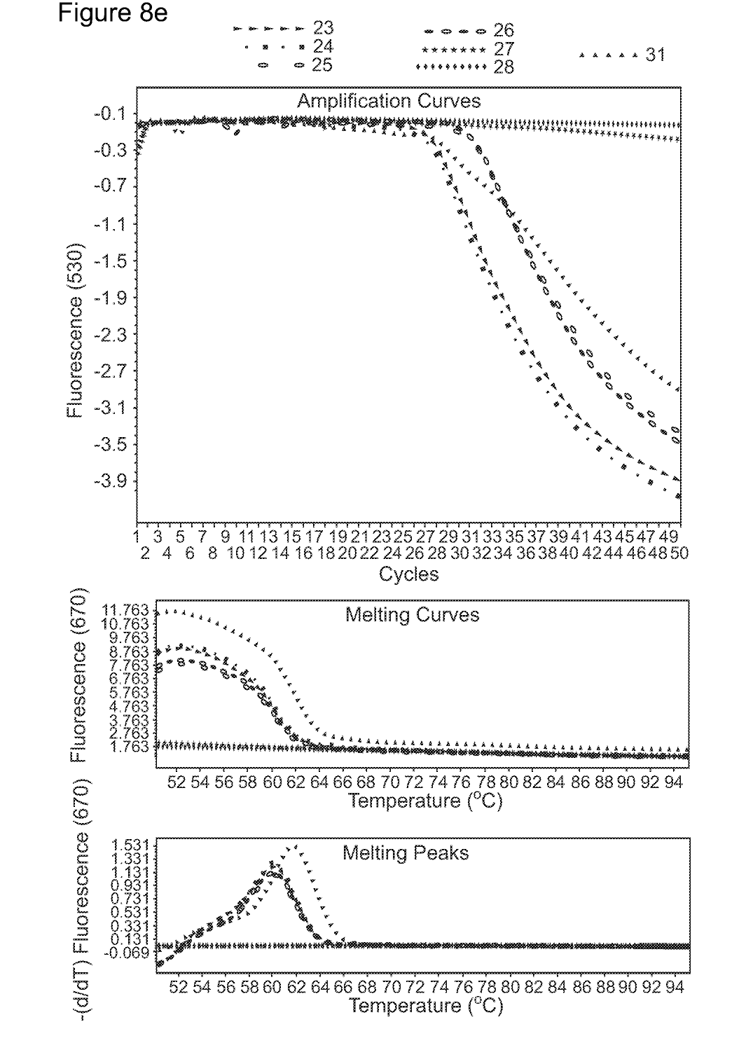

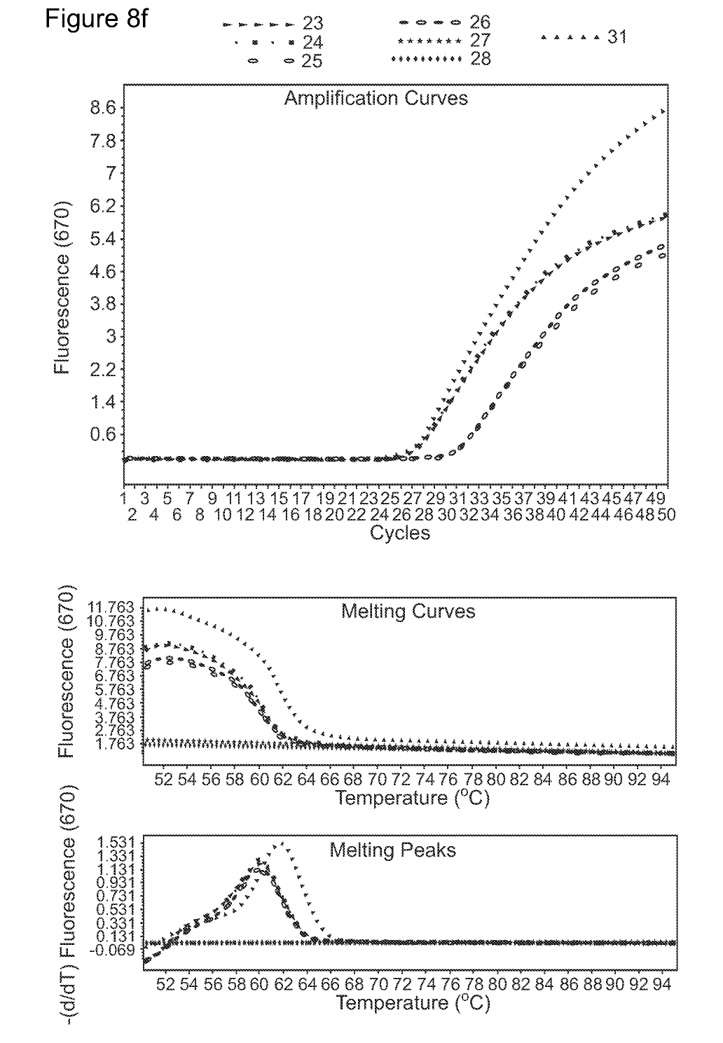

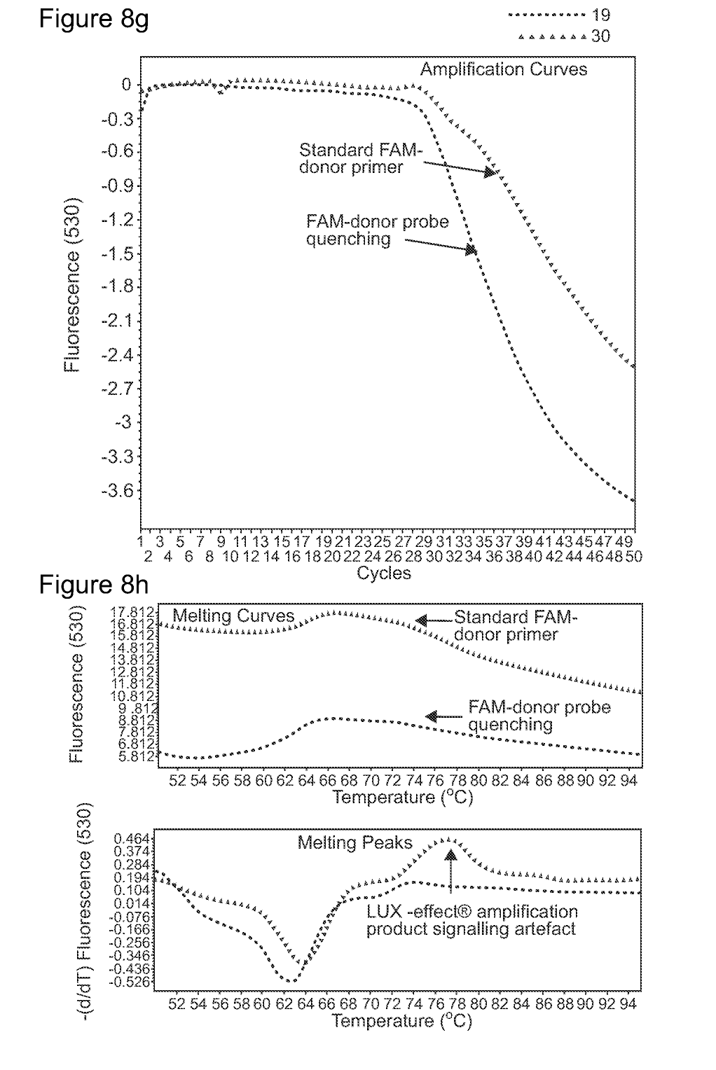

Although signal yield was lower, the FRET relationship between the donor probe and reporter primer did not generate the proximal quenching effect seen in the other trans signalling embodiments (see FIGS. 8g+h for example data).

Discussion

Method 1: Labelling Position (internal vs 5′ end 6FAM-labelled primer comparison) The signal of the reporter fluorophore was reduced by 30-40%, which was lower than expected, although still clearly detectable. This suggests that in the trans embodiment signalling chemistry is less spatially constrained by the nucleic acid configuration than the cis dual hybridisation approach, where two probes hybridise to the same nucleic acid strand. Notably, 5′ end-labelled 6FAM oligonucleotides have a lower manufacturing cost than internally labelled equivalents. The end-labelling position of 6FAM within the donor primer reduced, but did not eliminate, the induced proximal quenching from label incorporation into amplicon/ dimer species.

Method 2: Labelling Position (5′ end-labelled 6FAM-primer with molecular spacer) The addition of a linker did appear to reduce the loss in signal when locating 6FAM at the 5′ end of the primer giving a 15-25% reduction compared to a 30-40% without the spacer. This suggests that Dexter quenching does contribute to energy transfer in this configuration. The linker also reduced (but did not eliminate) the proximal quenching effect suggesting that the charge dissipation effects still occurred.

Method 3: Probe label switch (5′ TYE665-labelled primer+3′ 6FAM-labelled probe) Switching the signalling moieties to give a TYE665 reporter (acceptor) on the primer and the 6FAM (donor) on the probe resulted in a 30-40% reduction in reporter signal yield. The inventors believe that this reduction was due to the increased distance required for FRET rather than a direct effect of reversing the labels (as this primer label is now at the 5′ end rather than internal). The switching successfully eliminated the proximal quenching effects.

TABLE 7
Summary of PCR & Melt Analysis Data

| | | | Average | | |
|---|---|---|---|---|---|
| | | Average | Melt Tm | ΔTm | |
| | | PCR CT | WT DNA | standard | |
| Modification | Target Conc (copies/μl) | WT DNA | (° C.) | mix (° C.) | Remarks |
| MIX 1B | 1e4 WT DNA | 26.91 | 68.02 | — | Standard |
| TAMH5N1MLF1 (FAM) + | 1e3 WT DNA | 30.77 | 67.16 | — | H5N1 |
| TAMMLH5N1_H274Y (TYE665) | 1e4 SEQ ID NO 1 DNA | 27.79 | 64.17 | −3.85 | TRANS-DH |
| | 1e3 SEQ ID NO 1 DNA | 31.05 | 64.33 | −2.83 | assay |
| | 1e4 SEQ ID NO 2 DNA | 26.36 | 62.08 | −5.94 | |
| | 1e3 SEQ ID NO 2 DNA | 30.16 | 62.1 | −5.92 | |
| MIX 1C | 1e4 WT DNA | 27.21 | 67.81 | −0.21 | Forward |
| TAMH5N1MLF15′ (FAM) + | 1e3 WT DNA | 30 | 68.02 | 0.86 | primer 5′-end |
| TAMMLH5N1_H274Y (TYE665) | 1e4 SEQ ID NO 1 DNA | 27.84 | 64.5 | 0.33 | labelled with |
| | 1e3 SEQ ID NO 1 DNA | 30.27 | 64.35 | 0.02 | FAM |
| | 1e4 SEQ ID NO 2 DNA | 26.59 | 61.99 | −0.09 | |
| | 1e3 SEQ ID NO 2 DNA | 30 | 62.07 | −0.01 | |
| MIX 2B | 1e4 WT DNA | 26.66 | 67.84 | −0.18 | Forward |
| TAMH5N1MLF15′SP (FAM) + | 1e3 WT DNA | 30.74 | 67.91 | 0.75 | primer 5′-end |
| TAMMLH5N1_H274Y (TYE665) | 1e4 SEQ ID NO 1 DNA | 26.58 | 64.14 | −0.03 | labelled with |
| | 1e3 SEQ ID NO 1 DNA | 30.62 | 64.05 | −0.28 | FAM + |
| | 1e4 SEQ ID NO 2 DNA | 25.87 | 61.98 | −0.1 | internal C3 |
| | 1e3 SEQ ID NO 2 DNA | 29.06 | 62.02 | −0.08 | spacer |
| MIX 2C | 1e4 WT DNA | 27.25 | 66.45 | −1.57 | FAM-labeled |
| TAMH5N1MLF1 (TYE665) + | 1e3 WT DNA | 30.66 | 66.53 | −0.63 | probe with |
| TAMMLH5N1_H274Y (FAM) | 1e4 SEQ ID NO 1 DNA | 27.95 | 63.08 | −1.09 | TYE665 |
| | 1e3 SEQ ID NO 1 DNA | 31.15 | 63.31 | −1.02 | labeled |
| | 1e4 SEQ ID NO 2 DNA | 26.45 | 60.53 | −1.55 | primer (dye |
| | 1e3 SEQ ID NO 2 DNA | 30.12 | 60.45 | −1.65 | switch) |
| | 1e3 WT DNA | 29.74 | 63.95 | −3.21 | |
| | 1e4 SEQ ID NO 1 DNA | 25.94 | 59.59 | −4.58 | |
| | 1e3 SEQ ID NO 1 DNA | 27.76 | 59.67 | −4.66 | |
| | 1e4 SEQ ID NO 2 DNA | 26 | 64.96 | 2.88 | |
| | 1e3 SEQ ID NO 2 DNA | 31.46 | 64.99 | 2.89 | |

1. A system for detecting a target nucleic acid molecule of a particular sequence in a sample, said system comprising
i. an oligonucleotide primer complementary to said target nucleic acid molecule, which primer has no internal complementarity, is able to amplify said target sequence and carries a first label linked to said oligonucleotide at its 5′ end; and
ii. an oligonucleotide probe which carries a second label that is able to interact with said first label to produce a detectable signal, wherein the oligonucleotide probe binds an extension product of said primer such that the first and second label can interact to produce a detectable signal.

2. The system according to claim 1 wherein the first label is linked to the 5′ end of the primer by way of a spacer group.

3. The system according to claim 2 wherein the spacer is a 3C spacer, an octandiol, hexandiol or polyethylene glycol linker.

4. The system according to claim 1 wherein one of the first or second labels comprise a donor molecule of a FET or FRET signalling system and the other is an acceptor molecule of the FET or FRET signalling system.

5. The system according to claim 4 wherein the donor molecule is fluorescein or a fluorescein derivative.

6. The system according to claim 4 wherein the acceptor molecule is a TYE™ dye, Cy3, Cy3.5, Cy5, Cy5.5, Texas Red, Rox, Rhodamine, TYE563, TEX615, TYE 665, TYE700 or TYE705.

7. The system according to claim 4 wherein the donor molecule is attached at the 5′ end of the primer and the acceptor molecule is attached to the 3′ end of the probe.

8. The system according to claim 4 wherein the acceptor molecule is attached to the 5′ end of the primer and the donor molecule is attached to the 3′ end of the probe.

9. The system according to claim 1 which is adapted for use in a trans dual hybridisation assay.

10. The system according to claim 9 wherein the said assay comprises a multiplex arrangement.

11. The system according to claim 1 wherein the said oligonucleotide primer is a forward or reverse primer for use in an amplification reaction, and the system further comprises a second oligonucleotide primer able to act as the reverse or forward primers respectively, in the amplification of said target nucleic acid molecule.

12. The system according to claim 11 wherein the amplification reaction is a polymerase chain reaction.

13. The system according to claim 1 wherein the target nucleic acid molecule is derived from a microorganism.

14. The system according to claim 13 wherein the microorganism is a virus.

15. The system according to claim 14 wherein the virus is an influenza virus.

16. The system according to claim 15 wherein said influenza virus is type H1 N1 or H5N1.

17. The system according to claim 15 wherein the target region codes for H274Y in the neuraminidase gene of influenza virus.

18. A method for detecting the presence or amount of a target nucleic acid molecule comprising:
a) providing a sample containing or suspected of containing said nucleic acid molecule;
b) exposing the sample to a labelled oligonucleotide primer of the system according to claim 1 under conditions that enable annealing of said primer to any target nucleic acid molecule present in the sample, and extension of any annealed primer;
c) exposing any nucleic acid produced in step b) to a labelled oligonucleotide probe of the system under conditions that enable annealing of the probe to any complementary nucleic acid;
d) detecting an interaction between said first and second labels; and
e) relating this to the presence or amount of the target nucleic acid molecule in the sample.

19. The method according to claim 18 which is a nucleic acid amplification reaction.

20. The method according to claim 19 wherein the nucleic acid amplification reaction is a polymerase chain reaction.

21. The method according to claim 19 wherein the oligonucleotide probe of the system is present in the sample throughout the amplification reaction.

22. The method according to claim 21 wherein the signal from the sample is monitored throughout the amplification reaction.

23. The method according to claim 18 wherein the signal from the sample is monitored whilst the temperature is changed to provide an annealing or destabilisation temperature for a duplex formed between the oligonucleotide probe and an extension product of said oligonucleotide primer.

24. The method according to claim 18 which is carried out in a multiplex arrangement.

25. A kit for detecting the presence or amount of a target nucleic acid molecule, said kit comprising the system according to claim 1 and at least one reagent necessary to carry out a primer extension reaction.

26. (canceled)

27. An oligonucleotide primer for use in the system according to claim 1.

28. The oligonucleotide primer according to claim 27 which has no internal complementarity and carries a label linked to the oligonucleotide at its 5′ end.
